(19) United States
(12) Patent Application Publication
(10) Pub. No.: US 2007/0087974 A1
(43) Pub. Date: Apr. 19, 2007

(54) TREATMENT OR PREVENTION OF RESPIRATORY VIRAL INFECTIONS WITH IMMUNOMODULATOR COMPOUNDS

(75) Inventors: Cynthia W. TUTHILL, Menlo Park, CA (US); Alfred R. RUDOLPH, Los Altos Hills, CA (US); Alexander A. KOLOBOV, Sestroretsk (RU); Andrey S. SIMBIRTSEV, St. Petersburg (RU); Aleksandr V. PETROV, St. Petersburg (RU)

Correspondence Address: ROTHWELL, FIGG, ERNST & MANBECK, P.C., 1425 K STREET, N.W., SUITE 800, WASHINGTON, DC, 20005, US

(73) Assignee: SCICLONE PHARMACEUTICALS, INC., San Mateo, CA (US)

(21) Appl. No.: 11/558,281

(22) Filed: Nov. 9, 2006

Related U.S. Application Data
(63) Continuation of application No. PCT/US05/16595, filed on May 12, 2005.
(60) Provisional application No. 60/570,941, filed on May 14, 2004.

(51) Int. Cl. A61K 38/04; A61K 31/405

(52) U.S. Cl. 514/19; 514/419

(57) ABSTRACT

An immunomodulatory compound is administered to a patient having, or at risk of a respiratory viral infection.

### CROSS REFERENCE TO RELATED APPLICATIONS

This application is continuation of application PCT/US2005/016595, filed May 12, 2005, which claims priority to provisional application 60/570,941, filed May 14, 2004.

### BACKGROUND OF THE INVENTION

1. Field of the Invention

The present invention relates to the field of treatment or prevention of respiratory viral infections.

2. Description of the Background Art

Respiratory viral infections affect hundreds of millions of people every year.

There are three types of influenza viruses, A, B and C. All three types infect humans. However, type A is of the most concern and it infects many other animal species. Antigenic drift variants of classical human influenza A viruses re-emerge annually and infect approximately 500 million people during a moderate year. Periodically new antigenetically distinct viruses emerge and these antigenetic shift variants have the potential to cause pandemics that rapidly kill millions of people. Interspecies transmission is critical to the evolution and pathogenesis of antigenetic shift viruses with pandemic potential.

Coronaviruses infect humans, other mammals and avian species. Severe Acute Respiratory Syndrome (SARS) is caused by a coronavirus that initially emerged in China and spread to 30 other countries. SARS is an unusual coronavirus in that it contains neuramidase, which is normally found in influenza viruses. SARS virus may be described as an orthomyxovirus-coronavirus hybrid. The SARS virus and the virus that caused the 1918 Influenza Pandemic share a common gene sequence. Both viruses share the initial sequence “MNPNQKIITIGS”, indicating that they may be related. Both coronaviruses and orthomyxoviruses are known to infect animals, birds and humans. Both viruses have the ability to cross over from animals to humans.

There remains a need in the art for methods of treatment or prevention of respiratory viral infections.

### SUMMARY OF THE INVENTION

In accordance with the present invention, a method of treatment or prevention of a respiratory viral infection in a subject comprises administering to said subject an effective amount of an immunomodulator compound of formula A:

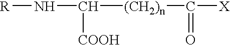

In Formula A, n is 1 or 2, R is hydrogen, acyl, alkyl or a peptide fragment, and X is an aromatic or heterocyclic amino acid or a derivative thereof. Preferably, X is L-tryptophan or D-tryptophan.

### BRIEF DESCRIPTION OF THE DRAWINGS

FIG. 1 shows survival of influenza-infected mice, and compares SCV-07-treated animals with control animals.

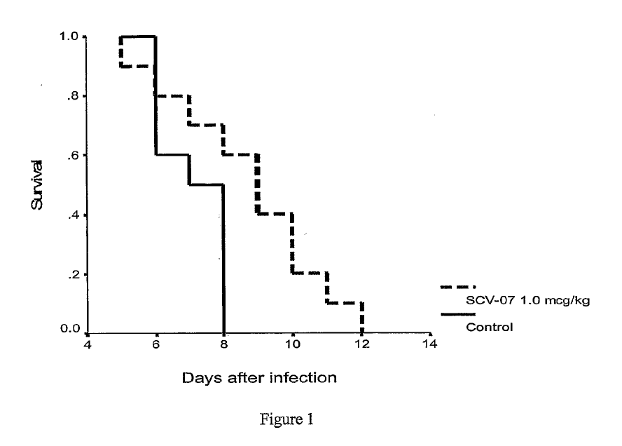

### DETAILED DESCRIPTION OF THE INVENTION

In accordance with one embodiment, the present invention relates to treatment or prevention of respiratory viral infections by administering an immunomodulator compound to a subject.

In accordance with another embodiment, the invention relates to treatment or prevention of coronavirus infection by administering an immunomodulator compound to a subject.

In accordance with a further embodiment, the invention relates to treatment or prevention of Severe Acute Respiratory Syndrome (SARS) in a subject by administering an immunomodulator compound.

In accordance with still a further embodiment, the invention relates to treatment or prevention of influenza in a subject by administering an immunomodulator compound.

In accordance with yet another embodiment, the invention relates to treatment or prevention of orthomyxovirus-coronavirus hybrid infection by administering an immunomodulator compound to a subject.

Preferably the subject is mammalian, most preferably the subject is a human patient.

Administration for prevention can be to persons at high risk because of contact with suspected disease carriers, or in carriers who are asymptomatic.

Immunomodulator compounds in accordance with the present invention, comprise immunomodulators of Formula A:

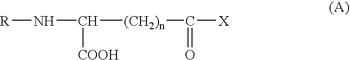

In Formula A, n is 1 or 2, R is hydrogen, acyl, alkyl or a peptide fragment, and X is an aromatic or heterocyclic amino acid or a derivative thereof. Preferably, X is L-tryptophan or D-tryptophan.

Appropriate derivatives of the aromatic or heterocyclic amino acids for “X” are: amides, mono-or di-(C₁-C₆) alklyl substituted amides, arylamides, and (C₁-C₆) alkyl or aryl esters. Appropriate acyl or alkyl moieties for “R” are: branched or unbranched alkyl groups of 1 to about 6 carbons, acyl groups from 2 to about 10 carbon atoms, and blocking groups such as carbobenzyloxy and t-butyloxycarbonyl. Preferably the carbon of the CH group shown in Formula A has a stereoconfiguration, when n is 2, that is different from the stereoconfiguration of X.

Preferred embodiments utilize compounds such as γ-D-glutamyl-L-tryptophan, γ-L-glutamyl-L-tryptophan, γ-L-glutamyl-Nᵢₙ-formyl-L-tryptophan, N-methyl-γ-L-glutamyl-L-tryptophan, N-acetyl-γ-L-glutamyl-L-tryptophan, γ-L-glutamyl-D-tryptophan, β-L-aspartyl-L-tryptophan, and β-D-aspartyl-L-tryptophan. Particularly preferred embodiments utilize γ-D-glutamyl-L-tryptophan, sometimes referred to as SCV-07. These compounds, methods for preparing these compounds, pharmaceutically acceptable salts of these compounds and pharmaceutical formulations thereof are disclosed in U.S. Pat. No. 5,916,878, incorporated herein by reference.

The Formula A compounds may be administered as dosages in the range of about 0.001-10 mg. Dosages may be administered one or more times per week, preferably on a daily basis, with dosages administered one or more times per day. The dosages may be administered by intramuscular injection, although other forms of injection and infusion may be utilized, and other forms of administration such as oral or nasal inhalation or oral ingestion may be employed.

In preferred embodiments, the compounds of Formula A are administered at a dosage within a range of about 0.01-10 mg, more preferably at a dosage of about 0.1-1 mg.

Dosages may also be measured in micrograms per kilogram subject body weight, with dosages in the range of about 0.01-100 micrograms per kilogram, more preferably within the range of about 0.1-10 micrograms per kilogram, and most preferably at about 1 microgram per kilogram.

Included are biologically active analogs having substituted, deleted, elongated, replaced, or otherwise modified portions which possess bioactivity substantially similar to that of SCV-07, e.g., an SCV-07 derived peptide having sufficient homology with SVC-07 such that it functions in substantially the same way with substantially the same activity as SCV-07.

Administration can be by any suitable method, including orally, by injection, periodic infusion, continuous infusion, and the like.

According to one embodiment, a Formula A compound may be administered to a patient in need of immune stimulation so as to substantially continuously maintain an immune stimulating-effective amount of the Formula A compound in the patient's circulatory system during a treatment or prevention period. Although much longer treatment periods are contemplated in accordance with the present invention, embodiments of the invention include substantially continuously maintaining an immune stimulating-effective amount of the Formula A compound in the patient's circulatory system during treatment periods of at least about 6, 10, 12 hours, or longer. In other embodiments, treatment periods are for at least about a day, and even for a plurality of days, e.g., a week or longer. However, it is contemplated that treatments, as defined above, in which immune stimulating-effective amounts of the Formula A compound are substantially continuously maintained in the patient's circulatory system, may be separated by non-treatment periods of similar or different durations.

In accordance with one embodiment, the Formula A compound is continuously infused into a patient, e.g., by intravenous infusion, during the treatment period, so as to substantially continuously maintain an immune stimulating-effective amount of the Formula A compound in the patient's circulatory system. The infusion may be carried out by any suitable means, such as by minipump.

Alternatively, an injection regimen of the Formula A compound can be maintained so as to substantially continuously maintain an immune stimulating-effective amount of the Formula A compound in the patient's circulatory system. Suitable injection regimens may include an injection every 1, 2, 4, 6, etc. hours, so as to substantially continuously maintain the immune stimulating-effective amount of the Immunomodulator compound peptide in the patient's circulatory system during the treatment period.

Although it is contemplated that during continuous infusion of the Formula A compound, administration will be for a substantially longer duration, according to one embodiment the continuous infusion of the Formula A compound is for a treatment period of at least about 1 hour. More preferably, continuous infusion is carried out for longer periods, such as for periods of at least about 6, 8, 10, 12 hours, or longer. In other embodiments, continuous infusion is for at least about one day, and even for a plurality of days such as for one week or more.

In some embodiments, the Formula A compound is present in a pharmaceutically acceptable liquid carrier, such as water for injection, saline in physiological concentrations, or similar.

The present invention also comprises administration of a physiologically active conjugate comprising a Formula A compound conjugated to a material which increases half-life of the Formula A compound in serum of a patient when said conjugate is administered to a patient. The material may be a substantially non-antigenic polymer. Suitable polymers will have a molecular weight within a range of about 200-300,000, preferably within a range of about 1,000-100,000, more preferably within a range of about 5,000-35,000, and most preferably within a range of about 10,000-30,000, with a molecular weight of about 20,000 being particularly preferred.

The polymeric substances included are also preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Among the substantially non-antigenic polymers, mono-activated, alkyl-terminated polyalkylene oxides (PAO's), such as monomethyl-terminated polyethylene glycols (mPEG's) are contemplated. In addition to mPEG, C1-4 alkyl-terminated polymers may also be useful.

As an alternative to PAO-based polymers, effectively non-antigenic materials such as dextran, polyvinyl pyrrolidones, polyacrylamides, polyvinyl alcohols, carbohydrate-based polymers and the like can be used. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

The polymer may be straight-chain or branched. Polyethylene glycol (PEG) is a particularly preferred polymer.

The polymer can be conjugated to the Formula A compound by any suitable method. Exemplary methods for conjugating polymers to peptides are disclosed in U.S. Pat. Nos. 4,179,337, 4,766,106, 4,917,888, 5,122,614 and 6,177,074, as well as PCT International Publication No. WO 95/13090, all of which are incorporated herein by reference. Polymer(s) can be conjugated at one or a plurality of sites.

Effective amounts of Formula A compound can be determined by routine dose-titration experiments.

The above dosages reflect only the Formula A compound present in the composition, and not the weight of the polymer conjugated thereto.

Conjugation of a polymer to a Formula A compound in accordance with the present invention substantially increases the plasma half-life of the peptide.

The Formula A compound also can be administered with an interferon, such as interferon alpha, wherein interferon alpha-2b is preferred. Suitable dosages of interferon alpha-2b may be in the range of about 1-3 MU.

The Formula A compound also can be administered with other immune stimulators or antiviral agents.

The invention is further illustrated by the following example, which is not intended to be limiting.

### EXAMPLE 1

The toxicogenic type of Influenza virus A—A/Achi/1/68 (H3N2) was used. To produce the virus two consecutive passages in 10-11 day chicken embryos were made. An ampoule with lyophilized Influenza virus from the collection of Pasteur's Research Institute (St-Petersburg, Russia) was diluted in 0.5 ml saline to obtain virus-containing fluids with 10⁻³ and 10⁻⁵ dilutions. These fluids were further used for inoculation of 10 embryos. Inoculated embryos were incubated for 48 hours at 37° C. and then cooled at 14° C. for 18-20 hours. Virus-containing fluid from each embryo was titrated from 1:2 to 1:4096 to determine the hemolyzing activity in 1% suspension of chicken erythrocytes. For the next passage specimens with at least 1:256 titer were selected and diluted 10⁻³ and 10⁻⁵. Each of these fluids was used for inoculation of another 10 embryos. Later the procedure was repeated once more. Virus-containing fluid with hemagglutinin titers 1:512-1:1024 was used to determine LD₁₀₀ in a preliminary experiment.

Specific pathogen-free outbred CFW female mice aged 4 weeks were purchased from Puschino animal facility (Puschino, Moscow District, Russia) and kept in air-conditioned and pathogen-free room with temperature of 21±2□C. and humidity of 55±10%. They were given standard laboratory chow and tap water ad libitum.

The mice were divided into four groups of 9-10 animals each and treated with 0.1, 1.0 or 10.0 mg/kg SCV-07 in 0.2 ml PBS per os for 5 days. The control group was treated with 0.2 ml PBS. Three days after the last application all mice were intranasally treated with LD₁₀₀ of virus-containing fluid (50 ml in each nostril) under short ether anesthesia and later observed daily for 14 days.

The comparison of survival curves between control and SCV-07-treated groups was performed using Log Rank test.

### RESULTS

The raw survival data for all groups of mice is shown in table 1 and medians of survival in table 2.

TABLE 1

| | SCV-07 | Number of animals died at indicated day after infection | | | | | | | | | | | | | | # of mice |
|---|---|---|---|---|---|---|---|---|---|---|---|---|---|---|---|---|
| # | (mg/kg) | 1 | 2 | 3 | 4 | 5 | 6 | 7 | 8 | 9 | 10 | 11 | 12 | 13 | 14 | survived |
| 1 | 0 | — | — | — | — | — | 4 | 1 | 5 | — | — | — | — | — | — | 0 |
| | (PBs) | | | | | | | | | | | | | | | |
| 2 | 0.1 | — | — | — | — | — | 4 | 2 | 1 | 2 | — | — | — | — | — | 0 |
| 3 | 1.0 | — | — | — | — | 1 | 1 | 1 | 1 | 2 | 2 | 1 | 1 | — | — | 0 |
| 4 | 10.0 | — | — | — | — | — | 6 | 2 | 1 | — | — | — | — | — | — | 0 |

TABLE 2

| | SCV-07 | |
|---|---|---|
| # | (mg/kg) | Median survival (days) |
| 1 | 0 | 7.0 |
| | (PBS) | |
| 2 | 0.1 | 7.0 |
| 3 | 1.0 | 9.0* |
| 4 | 10.0 | 6.0 |

*p = 0.025 compared to PBS-treated group by Log Rank test

1. A method of treatment or prevention of a respiratory viral infection in a subject, said method comprising administering to a subject infected with a respiratory virus an effective amount an immunomodulator compound of Formula A, or said method comprising administering to a subject at risk of infection by coronavirus, orthomyxovirus-coronavirus hybrid or SARS virus an effective amount of the immunomodulator compound of Formula A, or said method consisting essentially of administering to a subject at risk of infection by a respiratory virus an effective amount of the immunomodulating compound of Formula A, 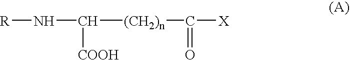
wherein, n is 1 or 2, R is hydrogen, acyl, alkyl or a peptide fragment, and X is an aromatic or heterocyclic amino acid or a derivative thereof.

2. The method of claim 1, wherein X is L-tryptophan or D-tryptophan.

3. The method of claim 1 wherein said compound is SCV-07.

4. The method of claim 1 wherein said respiratory viral infection is a coronavirus infection.

5. The method of claim 1 wherein said respiratory viral infection is an influenza infection.

6. The method of claim 1 wherein said respiratory viral infection is an orthomyxovirus-coronavirus hybrid infection.

7. The method of claim 1 wherein said infection is a SARS virus infection.

8. The method of claim 1 wherein said infection is an influenza virus infection.

9. The method of claim 1 wherein said compound is administered at a dosage within a range of about 0.1-10 mg.

10. The method of claim 1 wherein said compound is administered at a dosage within a range of about 0.1-1 mg.

11. The method of claim 1 wherein said compound is administered at a dosage within a range of about 0.01-100 micrograms per kilograms subject body weight.

12. The method of claim 1 wherein said compound is administered at a dosage within a range of about 0.1-10 micrograms per kilograms subject body weight.

13. The method of claim 12 wherein said compound is SCV-07.

14. The method of claim 4 wherein said compound is SCV-07.

15. The method of claim 5 wherein said compound is SCV-07.

16. The method of claim 6 wherein said compound is SCV-07.

17. The method of claim 7 wherein said compound is SCV-07.

18. The method of claim 8 wherein said compound is SCV-07.
